(19) United States
(12) Patent Application Publication
(10) Pub. No.: US 2021/0368805 A1
(43) Pub. Date: Dec. 2, 2021

(54) ANTIMICROBIAL MATERIAL

(71) Applicant: COPPER CLOTHING LIMITED, CATERHAM (GB)

(72) Inventor: RORY DONNELLY, CATERHAM (GB)

(21) Appl. No.: 17/309,464

(22) PCT Filed: Dec. 5, 2019
(86) PCT No.: PCT/GB2019/053433

§ 371 Date: May 28, 2021

(30) Foreign Application Priority Data
Dec. 5, 2018 (GB) 1819857.2

(51) Int. Cl. A01N 59/20; A61K 33/34; C22C 9/04

(52) U.S. Cl. CPC A01N 59/20; B22F 9/082; C22C 9/04; A61K 33/34

(57) ABSTRACT

The present invention relates to an antimicrobial material comprising a substrate and a metal component, wherein the metal component comprises chemically bonded copper and zinc. The invention also relates to methods of manufacturing the antimicrobial material described herein.

### FIELD OF INVENTION

The present invention relates to antimicrobial materials comprising copper and zinc incorporated into or coated on a substrate material, such as a polymer. The materials may be incorporated into a number of different products, including wound dressings, sanitary products and cleaning products. The invention also relates to methods of making the described antimicrobial materials.

### BACKGROUND

The antimicrobial properties of certain metals have been known for a substantial period of time. This unique property has been capitalised on in various industries, including agriculture and healthcare, in an attempt to control infection and contamination.

One metal commonly used in the healthcare setting is silver. The antimicrobial action of silver is dependent on the biologically active silver ion, resulting in irreversible damage to key enzyme systems within the cell membranes of pathogens, resulting in cell death. The most effective conditions for silver to act as an antimicrobial agent are those with higher temperatures and excess moisture. These conditions aid the ion-exchange reaction required for the release of silver ions. However, these particular conditions are rarely replicated in day-to-day healthcare settings, therefore limiting the efficacy of silver in controlling infection rates. In contrast, copper has been shown to display impressive antimicrobial efficacy in a broad range of environmental conditions.

Copper based materials are used in a wide-range of products, including wound dressings, sanitary protection products, toilet seats, clothing and footwear. Additionally, copper based materials are used in a number of medical settings, including in the treatment of arthritis and osteoporosis.

Copper is known to exert its actions in a number of ways; acting as a biocidal substance, enhancing microcirculation and reducing tissue inflammation at the site of injury. Additionally, the antimicrobial properties of copper are known to be an inherent feature, therefore representing a cost-effective and long-term solution to reducing infection rates.

The interest in using antimicrobial materials in the context of wound dressings is particularly prominent. A wound can fall into one of six types; abrasions, incisions, lacerations, punctures, avulsions or amputations. A particular challenge is the treatment of chronic wounds such as diabetic and pressure ulcers, resulting in prolonged disruption of the ‘barrier’ function of the skin, enhancing the possibility of contracting an infection.

The consequences of treating a wound ineffectively are manifold. These include enhanced hospitalisation rates, long-term disability, a reduction in workforce and an increased economic burden on society. Copper based materials have been shown to enhance the rate of wound healing via the mechanisms previously outlined, and as a result, increase the resolution of various infections. Additionally, silver based products have been reported to display much higher levels of toxicity compared to copper based products. For example, silver has been shown to lead to renal toxicity following topical application. However, the form of these copper based materials has varied widely, including the use of various copper alloys and copper salts.

Copper salts have been used for their antimicrobial properties in wound dressings. For example, US patent publication 2016/0220728 describes antimicrobial compositions comprising surface functionalised particles of low water solubility inorganic copper salts, or such copper salts infused into porous particles, and their application of the compositions for wound care.

Antimicrobial properties have also been associated with a copper-tin alloy. European patent publication EP 2 476 766 and US patent publication 2013/0323289 both describe antimicrobial raw materials comprising a substrate layer and a copper-tin alloy layer disposed on the substrate layer, suitable for use as wound dressing films and adhesive bandages. However, a number of issues are associated with this alloy, including skin discolouration when used in the context of a wound dressing.

Copper salts differ substantially to alloys in terms of the type of chemical bond involved between the two components. Alloys are produced via metallic bonding whereas copper salts are a result of ionic bonding between a base and an acid.

Copper based materials often involve an additional component, as opposed to using pure copper in isolation. Pure copper is a soft and malleable metal, limiting its utility in healthcare, agricultural and engineering industries. Conversely, copper alloys confer a number of desirable properties, including increased resistance to corrosion and enhanced strength. The increased resistance to corrosion and enhanced strength results in a more cost-effective and long-lasting material with wide-reaching applications in agriculture and engineering but such properties are not associated with advantages in healthcare applications. Copper takes on different properties when combined with different metals. For example, a copper-tin alloy results in a more brittle product compared to a copper-zinc alloy.

There is a need in the art for improved antimicrobial materials that can be used in wound dressings and sanitary products to reduce the incidence of local and systemic infection and accelerate healing.

The listing or discussion of an apparently prior-published document in this specification should not necessarily be taken as an acknowledgement that the document is part of the state of the art or is common general knowledge.

### SUMMARY OF INVENTION

In a first aspect, the present invention provides an antimicrobial material comprising a substrate and a metal component, wherein the metal component comprises chemically bonded copper and zinc.

In a second aspect, the present invention provides a method of manufacturing an antimicrobial material comprising a substrate and a metal component, wherein the metal component comprises chemically bonded copper and zinc, the method comprising the following steps:
a) combining copper and zinc to produce said metal component;
b) heating the metal component to a molten state;
c) disrupting said molten state with a high velocity gas, and;
d) combining the metal component with a substrate.

### DETAILED DESCRIPTION

The following description is presented to enable any person skilled in the art to make and use the invention. Various modifications to the disclosed embodiments will be readily apparent to those skilled in the art.

In a first aspect, the present invention provides an antimicrobial material comprising a substrate and a metal component, wherein the metal component comprises chemically bonded copper and zinc.

The term ‘antimicrobial material’ refers to a material having antimicrobial properties, for example biocidal or biostatic properties. In the context of the present invention, the term ‘biocidal’ is understood to mean a substance that can destroy, deter, render harmless or exert a controlling effect on a pathogenic organism, whereas the term ‘biostatic’ refers to a substance which can inhibit the growth or multiplication of an organism, for example a microorganism. It is envisaged that the present invention will be useful against any microorganism, for example any bacteria, virus and/or fungi. In particular, it is envisaged that bacteria in the Genus Staphylococcus and Klebsiella, fungi in the Genus Candida, and members of the Coronaviridae family, will be sensitive to the presently described materials.

The present invention provides materials with surprisingly high antimicrobial activity. Products, such as wound dressings, incorporating the materials of the invention will facilitate faster wound healing and reduced incidences of septicaemia and infection. The present invention is particularly useful in providing products to aid in the treatment of diabetic ulcers and other wounds that are slow to heal or close.

By ‘substrate’ we intend any suitable structural material to which the metal component can be incorporated, thereby providing a physical medium on or in which the metal component may be deployed. In preferred embodiments, the substrate is a foam. By ‘foam’ we intend a structure formed of the substrate material that has pockets of gas trapped within the substrate material. The foam may be a solid foam. The primary component of the solid foam may be a polymer-based material. The foam may alternatively be a liquid foam.

Examples of suitable polymers that may form the basis for the polymer-based material substrate include the synthetic polymers polyurethane and polypropylene and the naturally occurring matrix polymer collagen. The substrate may preferably be a polymer based hydrogel or a polymer based hydrocolloid. The polymer used in the hydrogel or hydrocolloid may be any polymer according to the disclosure. The term ‘polymer based hydrogels’ refers to polymer networks which are extensively swollen with water. Examples of the latter, which could be used in the present invention, include P-DERM® Hydrogels and Nanorestore Gels®. By ‘hydrocolloid’ we intend a substance which forms a gel in the presence of water.

By ‘chemically bonded’ we intend any lasting attraction between atoms, ions or molecules of copper and zinc as a result of ionic, covalent or metallic bonding. Accordingly, this may include copper alloys or copper compounds, including but not limited to copper salts and oxides.

Preferably, the metal component of the antimicrobial material comprises a copper-zinc alloy. An alloy is understood to be a mixture of two elements, one of which is a metal. In this instance, the copper-zinc alloy is understood to be a substitutional alloy, whereby the atoms of the two components may replace each other within the same crystal structure, creating a sea of delocalised electrons.

A skilled person would recognise that in order to produce the required alloy, elemental copper and zinc are mixed together in their molten form before solidifying as a new and distinct chemical entity. In one embodiment, it is envisaged that additional metals and compounds thereof, e.g. salts, may be incorporated into the material or metal component. These metals include, but are not limited to, zirconium, copper, zinc, silver, gold, palladium, platinum, iridium, aluminium, nickel, tungsten, molybdenum, tantalum, titanium, iodine. It is understood that the latter compounds may be additional components to the claimed material which contribute to a further enhancement of the antimicrobial properties of the material.

The use of an alloy, as opposed to the pure form of the metal or associated compounds, results in a number of advantageous properties compared to the use of pure copper. Specifically, a copper-zinc alloy benefits from the extra antimicrobial properties of zinc, excellent malleability/castability and high strength.

The particles of the metal component are expected to measure between 10-80 μm, with the preferred size being anywhere from 15-30 μm. A finely ground powder releases more ions compared to a course powder, the released ions of which may be responsible for the antimicrobial effect.

It is envisaged that the metal component will contain at least 60% copper. This formulation will have enhanced antimicrobial properties. Preferably, the metal component comprises 75-80% copper with a corresponding amount of 20-25% zinc. As outlined above, the metal component may additionally contain other element(s), compounds and salts thereof. These additions may confer beneficial properties to the claimed material. For example, additional components may further enhance the antimicrobial actions or allow for increased longevity of the claimed product.

In one embodiment of the present invention, the metal component may be interspersed throughout the substrate. By ‘interspersed’ we intend that the metal component is scattered between particles/molecules of the substrate material. Such a configuration could alternatively be described as ‘impregnation’. The metal component may be evenly or unevenly dispersed throughout the substrate material. A skilled person would understand that the degree of interspersion, dispersion and/or impregnation may depend on the polymer type used in the manufacture of the substrate material and/or the process used to apply the metal component to the substrate. In a further embodiment of the present invention, the metal component may be present as a coating layer on the surface of the substrate. Where a coating layer is present, it is expected that the coating will be arranged such that, in use, it comes into contact with a potentially contaminated surface/wound to exert its antimicrobial effect. The coating layer may be any thickness. Additionally, the coating layer is understood to be present on at least one surface of the substrate, but may be present on all substrate surfaces. The coating layer may either partially coat or completely coat a particular surface of the substrate. The degree of coverage of the coating layer will be dependent on the intended use of the claimed product.

It is envisaged that the substrate may be a polymer-based substrate. A polymer is a large molecule composed of smaller repeated subunits. Preferably, the substrate used in the present invention may include polyurethane, polypropylene, and/or collagen based polymers. The substrate may include polymer based hydrogels or polymer based hydrocolloids, according to the disclosure. Both thermosetting and thermoplastic polyurethanes may be suitable for use in the present invention. However, it is envisaged that any material suitable for stably maintaining the metal component may be used alone or in combination as substrates according to the present invention. For example, materials such as wool, cotton, leather, flax, ramie, silk, hemp, bamboo, jute, rayon, neoprene, elastane, rubber, polyester may be suitable as substrates as appropriate. In some instances, it is understood that the substrate may be a combination of different types of polymer. Such combinations may confer additional advantageous properties on the substrate for a desired purpose or to facilitate manufacture and storage. In particular, it is envisaged that alginates and cellulose could be incorporated into the substrate to enhance absorbency, flexibility and comfort. The skilled person will recognise that polymer-based hydrogels are particularly beneficial for use in wound dressings due to the presence of hydrophilic functional groups. This feature enables the control of moisture at a particular surface.

Preferably, the substrate may include the following ingredients following manufacture (% calculated on the weight of the finished dry product):
a) Sodium carboxymenthylcellulose 7%
b) Surfactant 3%
c) Glycerine 18%
d) Citric Acid 3.5%, and
e) The selected polymer 68.5%

Examples of suitable surfactants include sodium stearate, dioctyl sodium sulfosuccinate and perfluorooctanesulfonate. Suitable surfactants may belong to any of the following groups: anionic, cationic, non-ionic or zwitterionic surfactants. The citric acid element may be substituted with other weak acids if required, for example, acetic acid, lactic acid and phosphoric acid. Part e) of the above list may be substituted with any of the aforementioned polymers. Preferably, the polymer of choice is used in isolation; however different polymers may be used in combination if the end antimicrobial agent is deemed more effective and remains 68.5% of the substrate composition.

Preferably, 3-15% of the substrate by weight consists of the metal component. Also envisaged is the inclusion of further additives to the material to improve the antimicrobial properties, if required. These additives may include chelating agents, magnesium sulphate and/or a copper peptide. These additives may be incorporated into the substrate at 0.1 to 1% by weight, for example about 0.5% by weight. The term “chelating agent” is used to describe a substance that can form several bonds with a single metal ion thus forming a more stable complex. A skilled person would recognise the action of such substances could enhance the antimicrobial properties.

The present invention may be effective when it comes into contact with any contaminated surface. In a preferred embodiment, the present invention may be incorporated into a wound dressing suitable for application to the surface of human or animal skin of various anatomical locations. The antimicrobial material is preferably breathable. By ‘breathable’, it is intended that air flow to a wound or other surface to be treated is maintained. In the application to wound dressings, the ability to allow the wound to dry, or at least not swelter, is envisaged to further enhance the healing process. Preferably, the material has pores spaced throughout to facilitate breathability. A skilled person would understand how to arrange such pores dependent on the size and application of the material.

The present invention provides a high level of antimicrobial activity and therefore has wide-reaching applications. The present invention includes an infection control product comprising the antimicrobial material of the invention. Such a product may have utility in the healthcare setting, most often as a medical material. By ‘infection control product’ we intend any product that treats, prevents or attenuates the development and/or spread of infections. Examples of such products include wound dressings, bandages, medical devices, drug containers and personal protective clothing for infection protection.

It is envisaged that one application of the present invention may be the addition of the metal component to a hydrocolloid material for the treatment of sloughy wounds. A sloughy wound is one where necrotic tissue is separating itself from the wound site. These types of wound are known to exude liquid from said wound and therefore the use of a hydrocolloid dressing to convert this liquid into a gel form will be particularly beneficial.

The invention also provides a garment or household product comprising the antimicrobial product of the present disclosure. By ‘household product’ we intend any products typically used within a home, such as cleaning products and/or clothing. By ‘garment’ we intend any item which can be worn on any anatomical location of the body. Examples of garments that the present invention may be applied include underwear (including socks, vests, stockings, pants) that would typically come into intimate contact with the skin of the wearer. Other garments such as shoes, scarfs, trousers, gloves, hats, aprons, sport or physiotherapy joint supports (for example knee sleeves, neck supports, supportive briefs etc.) may be provided with the material of the present invention incorporated. Further, household cleaning products such as sponges, wipes (disposable or re-usable) and towels are also included.

The invention also provides a hygiene product, such as a sanitary towel, comprising the antimicrobial material of the invention. By ‘hygiene product’ we intend any product which is primarily for personal use and is intended to come into intimate contact with the skin or a bodily orifice of the user. Application of the antimicrobial material of the invention to such products will aid in preventing any harmful build-up of any particular microorganism and reduce the possibility of sepsis. Examples such hygiene products where the invention may be incorporated include curtains, bedding, cleaning products, sanitary towels, tampons and associated sanitary products, disposable nappies, incontinence pants and pads, clothing, footwear and means for transporting said products.

In a second aspect, the present invention provides a method of manufacturing an antimicrobial material comprising a substrate and a metal component, wherein the metal component comprises chemically bonded copper and zinc. The method comprises the steps of a) combining copper and zinc to produce said metal component; b) heating the metal component to a molten state; c) disrupting said molten state with a high velocity gas, and; d) combining the disrupted metal component with a substrate.

Thus, one method of producing the metal component of the invention may involve a plasma or gas atomisation process. It is envisaged that powdered forms of the metals may be used in the method of the invention but other forms could be appropriate as would be understood by a person of skill in the art.

It is envisaged that the plasma or gas atomisation process will result in a powdered form of the metal component, which can be combined with the substrate as appropriate, as would be understood by a person of skill in the art.

In a preferred embodiment, prior to the commencement of the plasma or gas atomisation process, the metal component may optionally be reduced in size via the use of a mechanical attrition process. By ‘mechanical attrition’ we intend any process by which the result is the gradual breakdown of the metal component into smaller elements. This process can be achieved via the use of a number of attrition devices, including but not limited to: attrition mill, horizontal mill, 1D vibratory mill, 3D vibratory mill and planetary mill. All of the above devices result in a reduction in size due to the energy imparted to the sample during impacts between the milling media. Thus, metallic forms copper and zinc may be ground down to an appropriate form for us in the methods of the invention.

Once the copper and zinc have been combined, the atomisation process may proceed. As would be understood by a person of skill in the art, the means of combining the copper and zinc may differ depending on the atomisation process to be utilised.

Plasma atomisation requires the metal component to be in a wire form to be used as a feedstock. This is typically a wire of an alloy of the metal component, as would be understood by a person of skill in the art. Contrary to conventional gas atomisation, plasma atomisation uses plasma torches to instantaneously melt and atomise the wire in a single step. A cooling tower is then used to convert the droplets formed into a spherical powder.

Alternatively, conventional gas atomisation may be used. This may involve the heating of the copper-zinc metal component to approximately 2000° C. to produce a molten state of said component. By ‘molten state’ we intend the liquid form of said metal component when exposed to high temperatures. As would be understood by a person skilled in the art, a high velocity gas stream may flow through an expansion nozzle, siphoning the molten metal component from an input chamber. Examples of gases that can be used in this process include nitrogen, argon, helium or air. The skilled person will recognise that it is possible to use more than one gas in this process and the preferred gas or gas mixture will be inert/unreactive. The choice of gas used will depend on the desired end disrupted metal (powder) characteristics. To provide a suitable metal component for use in materials of the invention, high velocities of inert gas may be required. A skilled person will recognise that the velocity required will differ depending on the gas used but are likely to be within the range of 100-2000 m/s. This process disrupts the liquid stream of molten metal and results in the production of fine particles, culminating in the desired powdered form of the metal component. Obtaining the powdered form via the above methods has a number of advantages; production of highly spherical particles, low oxygen content and adaptability to the production of copper and zinc. A skilled person will also recognise that alternative methods of producing the metal powder may exist which could be employed to achieve the same effect.

To produce the final antimicrobial material, the metal component is added to the substrate. Specifically, the metal powder is added in small quantities until the entirety of the product is transferred to the substrate. The resulting composition is mixed at room temperature (20-22° C.) for 2 hours at 350 rpm and subsequently allowed to solidify.

To create the hydrocolloid product, the hydrocolloid material is heated to 240° C. before the metal component in the adhesive can be added. The adhesive component is in gel form and may comprise 80% carboxymethyl cellulose and 20% adhesive. The hydrocolloid material and adhesive component are mixed extensively to ensure even distribution throughout the resulting material.

The present invention also provides a method of treating a wound infection comprising applying the antimicrobial material of the invention to the wound.

In order that the invention may be more clearly understood embodiments thereof will now be described by way of example.

### Example 1

Test of the antimicrobial material on two different strains of bacteria: Staphylococcus aureus and Klebsiella pneumoniae.

Each test organism was prepared to approximately 1×10⁵ colony forming units (CFU)/mL in 0.85% NaCl. For each sample, five replicates were inoculated with each test organism. The inocula were enumerated using pour plates of Tryptone Soya Agar (TSA) at the point of inoculation. The inoculated samples were held for 24 hours at 24° C.±1° C. and >95% humidity. Following the exposure time, the inoculated test pieces were aseptically removed to 9 ml diluent. This was vigorously shaken to ensure thorough resuspension of any remaining test organisms. The resulting suspension was plated out in TSALT (TSA supplemented with 0.3% soya lecithin and 3% Tween 80). Plates were incubated at 31° C.±1° C. for at least 5 days.

TABLE 1
Effect of 0% CuZn foam material on two types of bacteria:
Staphylococcus aureus and Klebsiella pneumoniae.
24-hour contact time

| | | Recovery per test | | | | Mean Log₁₀ | |
|---|---|---|---|---|---|---|---|
| | | piece (CFU) | | Log₁₀ reductions | | reductions | |
| | | S. aureus | K. | S. aureus | K. | S. aureus | K. |
| | | ATCC | pneumoniae | ATCC | pneumoniae | ATCC | pneumoniae |
| Sample: | Replicate | 6538 | NCO9633 | 6538 | NCO9633 | 6538 | NCO9633 |
| Inoculum | — | 1.1 × 10⁵ | 9.6 × 10⁴ | — | — | — | — |
| (zero | | | | | | | |
| time) | | | | | | | |
| 0% CuZn | 1 | 5.0 × 10³ | >1.0 × 10⁶ | 1.34 | 0 | 1.32 | 0 |
| Foam | 2 | 5.6 × 10³ | >1.0 × 10⁶ | 1.29 | 0 | | |
| | 3 | 4.7 × 10³ | >1.0 × 10⁶ | 1.37 | 0 | | |
| | 4 | 5.7 × 10³ | >1.0 × 10⁶ | 1.28 | 0 | | |
| | 5 | 5.0 × 10³ | >1.0 × 10⁶ | 1.34 | 0 | | |

TABLE 2
Effect of 3% CuZn foam material on two types of bacteria:
Staphylococcus aureus and Klebsiella pneumoniae.
24-hour contact time

| | | Recovery per test | | | | Mean Log₁₀ | |
|---|---|---|---|---|---|---|---|
| | | piece (CFU) | | Log₁₀ reductions | | reductions | |
| | | S. aureus | K. | S. aureus | K. | S. aureus | K. |
| | | ATCC | pneumoniae | ATCC | pneumoniae | ATCC | pneumoniae |
| Sample: | Replicate | 6538 | NCO9633 | 6538 | NCO9633 | 6538 | NCO9633 |
| Inoculum | — | 2.8 × 10⁵ | 2.4 × 10⁵ | — | — | — | — |
| (zero | | | | | | | |
| time) | | | | | | | |
| 3% CuZn | 1 | <10 | <10 | >4.45 | >4.38 | >4.45 | >4.38 |
| Foam | 2 | <10 | <10 | >4.45 | >4.38 | | |
| | 3 | <10 | <10 | >4.45 | >4.38 | | |
| | 4 | <10 | <10 | >4.45 | >4.38 | | |
| | 5 | <10 | <10 | >4.45 | >4.38 | | |

TABLE 3
Effect of 15% CuZn foam material on two types of bacteria:
Staphylococcus aureus and Klebsiella pneumoniae.
24-hour contact time

| | | Recovery per test | | | | Mean Log₁₀ | |
|---|---|---|---|---|---|---|---|
| | | piece (CFU) | | Log₁₀ reductions | | reductions | |
| | | S. aureus | K. | S. aureus | K. | S. aureus | K. |
| | | ATCC | pneumoniae | ATCC | pneumoniae | ATCC | pneumoniae |
| Sample: | Replicate | 6538 | NCO9633 | 6538 | NCO9633 | 6538 | NCO9633 |
| Inoculum | — | 2.8 × 10⁵ | 2.4 × 10⁵ | — | — | — | — |
| (zero | | | | | | | |
| time) | | | | | | | |
| 15% | 1 | <10 | <10 | >4.45 | >4.38 | >4.45 | >4.38 |
| CuZn | 2 | <10 | <10 | >4.45 | >4.38 | | |
| Foam | 3 | <10 | <10 | >4.45 | >4.38 | | |
| | 4 | <10 | <10 | >4.45 | >4.38 | | |
| | 5 | <10 | <10 | >4.45 | >4.38 | | |

For samples ‘3% CuZn Foam’ and ‘15% CuZn Foam’ both bacterial strains were seen to be reduced in number by >4 log over a 24-hour contact time. This was compared to the sample ‘0% CuZn Foam’, which displayed no significant antibacterial activity against either test organism.

### Example 2

Test of the Antimicrobial Material on the Fungus Candida albicans.

The test organism was prepared to approximately 1×10⁶ CFU/mL in 0.85% NaCl. For each sample, five replicate test pieces were inoculated with an appropriate volume of the test organism (Table 2). The inocula were enumerated using pour-plates of Sabouraud Dextrose Agar (SDA) at the point of inoculation. The inoculated samples were then placed in an incubator at 24° C.±1° C. for 1, 8 or 24 hours at >95% humidity. Following the required exposure times, the inoculated test pieces were aseptically removed to 9 mL of diluent. This was vigorously shaken to ensure thorough resuspension of any remaining test organisms. The resulting suspension was plated out in SDALT (SDA supplemented with 0.3% soya lecithin and 3% Tween 80). Plates were incubated at 24° C.±1° C. for at least five days. For the negative control, the samples were inoculated with an appropriate volume (Table 2) of sterile 0.85% NaCl and incubated and analysed in the same way as the test samples.

TABLE 4
Sample inoculum volumes

| Sample | Inoculum volume |
|---|---|
| 0% CuZn Foam | 1.0 mL |
| 2% CuZn Foam | 300 μL |
| 3% CuZn Foam | 1.25 mL |

TABLE 5
Effect of 0% CuZn Foam material on the fungus Candida albicans.
1, 8 or 24 hour contact times with Candida albicans ATCC 10231

| | | | | | | | | Log10 reductions | | |
|---|---|---|---|---|---|---|---|---|---|---|
| | | Recovery per test piece | | | Log10 reductions | | | of mean recovery | | |
| Sample: | Replicate | 1 hr | 8 hr | 24 hr | 1 hr | 8 hr | 24 hr | 1 hr | 8 hr | 24 hr |
| Inoculum | — | 1.2 × 10⁶ | | | — | | | — | | |
| (zero time) | | | | | | | | | | |
| Positive | — | 9.1 × 10⁵ | 9.6 × 10⁵ | 1.5 × 10⁶ | 0.12 | 0.10 | 0 | 0.12 | 0.10 | 0 |
| control | | | | | | | | | | |
| Negative | — | <10 | <10 | <10 | — | — | — | — | — | — |
| control | | | | | | | | | | |
| 0% CuZn | 1 | 5.7 × 10⁵ | 4.1 × 10⁵ | >10⁶ | 0.32 | 0.47 | <0.08 | 0.47 | 0.32 | <0.10 |
| Foam | 2 | 4.0 × 10⁵ | 6.0 × 10⁵ | >10⁶ | 0.48 | 0.30 | <0.08 | | | |
| | 3 | 4.3 × 10⁵ | 7.1 × 10⁵ | 7.4 × 10⁵ | 0.45 | 0.23 | 0.21 | | | |
| | 4 | 2.8 × 10⁵ | 5.9 × 10⁵ | >10⁶ | 0.63 | 0.31 | <0.08 | | | |
| | 5 | 3.8 × 10⁵ | 5.8 × 10⁵ | >10⁶ | 0.50 | 0.32 | <0.08 | | | |

TABLE 6
Effect of 2% CuZn Foam material on the fungus Candida albicans.
1, 8 or 24 hour contact times with Candida albicans ATCC 10231

| | | | | | | | | Log10 reductions | | |
|---|---|---|---|---|---|---|---|---|---|---|
| | | Recovery per test piece | | | Log10 reductions | | | of mean recovery | | |
| Sample: | Replicate | 1 hr | 8 hr | 24 hr | 1 hr | 8 hr | 24 hr | 1 hr | 8 hr | 24 hr |
| Inoculum | — | 3.6 × 10⁵ | | | — | | | — | | |
| (zero time) | | | | | | | | | | |
| Negative | — | <10 | <10 | <10 | — | — | — | — | — | — |
| control | | | | | | | | | | |
| 2% CuZn | 1 | 1.2 × 10² | 1.0 × 10¹ | <10 | 3.48 | 4.56 | >4.56 | 3.22 | >4.56 | >4.56 |
| Foam | 2 | 1.9 × 10² | <10 | <10 | 3.28 | >4.56 | >4.56 | | | |
| | 3 | 2.7 × 10² | <10 | <10 | 3.13 | >4.56 | >4.56 | | | |
| | 4 | 2.4 × 10² | <10 | <10 | 3.18 | >4.56 | >4.56 | | | |
| | 5 | 3.0 × 10² | <10 | 1.0 × 10¹ | 3.08 | >4.56 | 4.56 | | | |

TABLE 7
Effect of 3% CuZn Foam material on the fungus Candida albicans.
1, 8 or 24 hour contact times with Candida albicans ATCC 10231

| | | | | | | | | Log10 reductions | | |
|---|---|---|---|---|---|---|---|---|---|---|
| | | Recovery per test piece | | | Log10 reductions | | | of mean recovery | | |
| Sample: | Replicate | 1 hr | 8 hr | 24 hr | 1 hr | 8 hr | 24 hr | 1 hr | 8 hr | 24 hr |
| Inoculum | — | 1.5 × 10⁶ | | | — | | | — | | |
| (zero time) | | | | | | | | | | |
| Negative | — | <10 | <10 | <10 | — | — | — | — | — | — |
| control | | | | | | | | | | |
| 3% CuZn | 1 | 4.3 × 10⁵ | 1.0 × 10¹ | <10 | 0.55 | 5.18 | >5.18 | 0.66 | >3.98 | >5.18 |
| Foam | 2 | 3.9 × 10⁵ | <10 | 1.0 × 10¹ | 0.59 | >5.18 | 5.18 | | | |
| | 3 | 3.9 × 10⁵ | 7.0 × 10¹ | <10 | 0.59 | 4.33 | >5.18 | | | |
| | 4 | 1.7 × 10⁵ | 3.5 × 10² | 1.0 × 10¹ | 0.95 | 3.64 | 5.18 | | | |
| | 5 | 2.8 × 10⁵ | 3.6 × 10² | <10 | 0.73 | 3.62 | >5.18 | | | |

For samples ‘0% CuZn Foam’ no significant reduction in the numbers of Candida albicans was observed after 1, 8 or 24 hour contact times at 24° C. For sample ‘2% CuZn Foam’ a greater than 3 log reduction in the numbers of Candida albicans was observed after a contact time of 1 hour; a greater than 4 log reduction in the numbers of Candida albicans was observed after 8 hour or 24 hour contact times at 24° C. For sample ‘3% CuZn Foam’ no significant reduction in the numbers of Candida albicans was observed after a 1 hour contact time; a greater than 3 log reduction in the numbers of Candida albicans was observed after 8 hours at 24° C.; a greater than 5 log reduction in the number of Candida albicans was observed after 24 hours at 24° C.

### Example 3

Test of the Antimicrobial Material on the Bovine Corona Virus (BCV) Strain L9.

For the preparation of the material, pieces of 1×1 cm were cut in sterile conditions and after a folding step transferred to an Eppendorf cup. For preparation of test virus solution, U373 cells were cultivated. For virus production, BCV strain L9 was added to the prepared monolayer. After an incubation period of 24-48 hours, cells were lysed by a rapid freeze/thaw cycle. Cellular debris was removed and the supernatant was directly used as the test virus suspension. Infectivity was determined by means of end point dilution titration using the microtitre process. The virucidal activity of the treated material was evaluated by calculating the decrease in titre in comparison with the virucidal activity of the non-treated material.

TABLE 8
Virus titres (bovine coronavirus) in the 10 fold assay with treated (novel
green/white nylon copper infused fabric) and non-treated material (reference:
Tork Premium Special Tucher) after 60 minutes exposure time.

| | Dilutions (log₁₀) | | | | | | | | | |
|---|---|---|---|---|---|---|---|---|---|---|
| Product | 1 | 2 | 3 | 4 | 5 | 6 | 7 | 8 | 9 | 10 |
| Copper | ≤1.50 ± | ≤1.50 ± | ≤1.50 ± | ≤1.50 ± | ≤1.50 ± | ≤1.50 ± | ≤1.50 ± | ≤1.50 ± | ≤2.00 ± | ≤1.50 ± |
| material | 0.00 | 0.00 | 0.00 | 0.00 | 0.00 | 0.00 | 0.00 | 0.00 | 0.44 | 0.00 |
| Reference | 2.83 ± | 3.25 ± | 2.63± | 2.88 ± | 3.38 ± | 3.13 ± | 2.88 ± | 3.88 ± | 2.63 ± | 2.63 ± |
| material | 0.37 | 0.33 | 0.433 | 0.37 | 0.25 | 0.37 | 0.37 | 0.37 | 0.41 | 0.25 |

After a contact time of 60 minutes, only one material residual virus could be measured with the novel green/white nylon copper infused fabric. In contrast, examining the non-treated materials residual virus could be detected in all cases. The following mean values resulted: ≤1.55±0.04 (novel green/white nylon copper infused fabric) and 2.98±0.12 (reference). A difference of 1.43 log₁₀ steps between both materials was visible based on the 10 fold determinations after 60 minutes exposure time.

I claim:

1. An antimicrobial material comprising a substrate and a metal component, wherein the metal component comprises chemically bonded copper and zinc.

2. The antimicrobial material according to claim 1, wherein the copper and zinc form an alloy.

3. The antimicrobial material according to claim 1 or 2, wherein the metal component further includes:
(a) any metal selected from the group consisting of zirconium, silver, gold, palladium, platinum, iridium, aluminium, nickel, tungsten, molybdenum, tantalum, titanium, iodine and/or any alloys thereof; or
(b) one or more salts of any of zirconium, copper, zinc, silver, gold, palladium, platinum, iridium, aluminium, nickel, tungsten, molybdenum, tantalum, titanium and/or iodine.

4. (canceled)

5. The antimicrobial material according to claim 1, wherein the metal component comprises particles measuring 3-50 μm.

6. The antimicrobial material according to claim 1, wherein the metal component comprises at least 60% copper.

7. (canceled)

8. The antimicrobial material according to claim 1, wherein the metal component comprises 20-25% zinc.

9. The antimicrobial material according to claim 1, wherein the metal component is interspersed throughout the substrate and/or wherein the metal component forms a coating layer on a surface of the substrate.

10. The antimicrobial material according to claim 9, wherein the coating layer has antimicrobial properties and is arranged such that, in use, the surface comes into contact with a potentially contaminated surface.

11. The antimicrobial material according to claim 1, wherein the substrate comprises a polymer-based substrate.

12. (canceled)

13. The antimicrobial material according to claim 1, wherein 3-15% of said material by weight consists of the metal component.

14. The antimicrobial material according to claim 1, wherein the material further includes chelating compounds, magnesium sulphate and/or a copper peptide.

15. (canceled)

16. An infection control product, comprising the antimicrobial material of claim 1.

17. A garment or household product comprising the antimicrobial material of claim 1.

18. A hygiene product, comprising the antimicrobial material of claim 1.

19. A method of manufacturing an antimicrobial material comprising a substrate and a metal component, wherein the metal component comprises chemically bonded copper and zinc,
the method comprising the steps of:
a) combining copper and zinc to produce said metal component;
b) heating the metal component to a molten state;
c) disrupting said molten state with a high velocity gas, and;
d) combining the disrupted metal component with a substrate.

20. The method according to claim 19, wherein the metal component is reduced in size prior to step (b) using a mechanical attrition process.

21. The method according to claim 19, wherein step (c) results in a powdered form of said metal component.

22. The method according to claim 19, wherein the metal component is heated at step (b) to 2000 degrees centigrade.

23. An antimicrobial material, infection control product, garment, household product or hygiene product, comprising a substrate and a metal component, wherein the metal component comprises chemically bonded copper and zinc, obtainable by the method of claim 19.

24. A method of treating a wound infection comprising applying the antimicrobial material of claim 1 to the wound.
